(19) United States
(12) Patent Application Publication
(10) Pub. No.: US 2016/0029974 A1
(43) Pub. Date: Feb. 4, 2016

(54) SYSTEM AND METHOD FOR TRACKING BIOLOGICAL AGE OVER TIME BASED UPON HEART RATE VARIABILITY USING EARPHONES WITH BIOMETRIC SENSORS

(71) Applicant: JayBird LLC, Salt Lake City, UT (US)

(72) Inventors: JUDD ARMSTRONG, Parrearra (AU); STEPHEN DUDDY, Moama (AU)

(73) Assignee: JAYBIRD LLC, Salt Lake City, UT (US)

(21) Appl. No.: 14/871,908

(22) Filed: Sep. 30, 2015

Related U.S. Application Data
(63) Continuation-in-part of application No. 14/830,549, filed on Aug. 19, 2015.
Application No. 14/830,549 is a continuation-in-part of application No. 14/244,464, filed on Apr. 3, 2014.
Application No. 14/244,464 is a continuation-in-part of application No. 14/137,734, filed on Dec. 20, 2013.
Application No. 14/137,734 is a continuation-in-part of application No. 14/062,815, filed on Oct. 24, 2013.

(51) Int. Cl. A61B 5/00; A61B 5/145; A61B 5/107; G06F 19/00; A61B 5/0205

(52) U.S. Cl. CPC A61B 5/7278; G06F 19/345; G06F 19/3481; A61B 5/6817; A61B 5/743; A61B 5/7275; A61B 5/0205; A61B 5/4815; A61B 5/7246; A61B 5/746; A61B 5/4884; A61B 5/4866; A61B 5/1072; A61B 5/4872; A61B 5/14546; A61B 5/486; A61B 5/1118

(57) ABSTRACT

Systems and methods are provided for using earphones with biometric sensors to track a user's biological age over time. In one embodiment, the system includes earphones, including: speakers; a processor; and a heartrate sensor electrically coupled to the processor. In this embodiment, the system also includes a memory coupled to a processor and having instructions stored that, when executed by the processor: receive one or more user determinable biological age parameters including an actual age of the user; determine one or more system measurable biological age parameters based on signals generated by the heartrate sensor; calculate a biological age factor as a function of the user determinable biological age parameters and the system measurable biological age parameters; calculate the user's biological age as a function of the biological age factor and the user's actual age; and display on a display the user's biological age.

### CROSS-REFERENCE TO RELATED APPLICATIONS

This application is a continuation-in-part of and claims the benefit of U.S. patent application Ser. No. 14/830,549 filed Aug. 19, 2015, titled “Earphones with Biometric Sensors,” the contents of which are incorporated herein by reference in their entirety. This application is also a continuation-in-part of and claims the benefit of U.S. patent application Ser. No. 14/244,464 filed Apr. 22, 2014 titled “Systems and Methods for Tracking Biological Age over Time Based Upon Heart Rate Variability,” which is a continuation-in-part of and claims the benefit of U.S. patent application Ser. No. 14/137,734, filed Dec. 20, 2013, titled “System and Method for Providing a Smart Activity Score,” which is a continuation-in-part of U.S. patent application Ser. No. 14/062,815, filed Oct. 24, 2013, titled “Wristband with Removable Activity Monitoring Device,” the contents all of which are incorporated herein by reference in their entirety.

### TECHNICAL FIELD

The present disclosure relates to earphones with biometric sensors, and more particularly embodiments describe systems and methods for tracking biological age over time based upon heart rate variability using earphones with biometric sensors.

### DESCRIPTION OF THE RELATED ART

Previous generation movement monitoring and fitness tracking devices generally enabled only a tracking of activity that accounts for total calories burned. One issue with currently available fitness tracking devices is that they do not account for the performance state of the user in a scientific, user-specific way. Another issue is that currently available solutions do not account in a precise manner for the health and performance benefits of sustained activity.

Additionally, understanding the effects of physical activity on biological age is becoming more important to many health conscious consumers. Biological age is essentially a person's actual age weighted by factors effecting that person's longevity. For example, factors such as gender, ethnicity, health, eating habits, stress levels, sleep habits, and exercise habits may increase or decrease a person's life expectancy. Biological age may change over time based on changes to any of these factors.

### BRIEF SUMMARY OF THE DISCLOSURE

Embodiments of the present disclosure include systems and methods for tracking a user's biological age over time based upon heart rate variability using earphones with biometric sensors.

In one embodiment, a system for tracking a user's biological age over time, includes: a pair of earphones including: speakers; a processor; and a heartrate sensor electrically coupled to the processor, where the processor is configured to process electronic input signals from the heartrate sensor. The system also includes a memory coupled to a processor and having instructions thereon that, when executed by the processor, causes the system to: receive one or more user determinable biological age parameters including an actual age of the user; determine one or more system measurable biological age parameters based on signals generated by the heartrate sensor; calculate a biological age factor as a function of the user determinable biological age parameters and the system measurable biological age parameters; calculate the user's biological age as a function of the biological age factor and the user's actual age; and display on a display the user's biological age. In various embodiments, the system measurable biological age parameters include heart rate variability, and the heart rate variability is determined based on signals generated by the heartrate sensor.

In one embodiment, the heartrate sensor is an optical heartrate sensor protruding from a side of the earphone proximal to an interior side of a user's ear when the earphone is worn. In this embodiment, the optical heartrate sensor is configured to measure the user's blood flow and to output an electrical signal representative of this measurement to the earphones processor.

In one embodiment, the system displays on the display a plot of the user's biological age trend function. This embodiment may be implemented by: receiving input from the user specifying a time interval width and desired number of time intervals for which to track the user's biological age; calculating, for each time interval, an interval specific biological age; storing in a memory, for each time interval, the interval specific biological age; calculating a biological age trend function based on the interval specific biological ages; and displaying on the display a plot of the biological age trend function.

In embodiments, the pair of earphones include a motion sensor that generate signals representative of the user's motion. In various implementations of these embodiments, the one or more system measurable biological age parameters are determined based on the signals generated by the motion sensor. In these embodiments, the system measurable biological age parameters may include a sleep profile, an activity profile, a recovery score, an activity score, and/or a smart activity score generated based on the signals representative of the user's motion. In various implementations, the sleep profile may include an average sleep quality metric and an average sleep quantity metric; and the activity profile may include an average activity quality metric and an average activity quantity metric. In a particular implementation of these embodiments, the system may display on the display a lifestyle trend function temporally synchronous with the biological age trend function, where the lifestyle trend function is based on the activity profile.

In one embodiment, the user determinable biological age parameters further include at least two of: gender, ethnicity, eating habits, stress level, marriage profile, height, weight, body fat index, cholesterol level, blood pressure, and medical history. In a particular implementation of this embodiment, the system may determine one or more adjustments to the system measurable biological age factors and the user determinable biological age factors such that, if the adjustments are achieved, the user's biological age will match the user's actual age.

Other features and aspects of the disclosed method and system will become apparent from the following detailed description, taken in conjunction with the accompanying drawings, which illustrate, by way of example, the features in accordance with embodiments of the disclosure. The summary is not intended to limit the scope of the claimed disclosure, which is defined solely by the claims attached hereto.

### BRIEF DESCRIPTION OF THE DRAWINGS

The present disclosure, in accordance with one or more various embodiments, is described in detail with reference to the following Figures. The Figures are provided for purposes of illustration only and merely depict typical or example embodiments of the disclosure.

FIG. 1 illustrates an example communications environment in which embodiments of the disclosed technology may be implemented.

FIG. 2A illustrates a perspective view of exemplary earphones that may be used to implement the technology disclosed herein.

FIG. 2B illustrates an example architecture for circuitry of the earphones of FIG. 2A.

FIG. 3A illustrates a perspective view of a particular embodiment of an earphone, including an optical heartrate sensor, in accordance with the disclosed technology.

FIG. 3B illustrates a side perspective view of placement of the optical heartrate sensor of the earphones of FIG. 3A when they are worn by a user.

FIG. 3C illustrates a frontal perspective view of placement of the optical heartrate sensor of the earphones of FIG. 3A when they are worn by a user.

FIG. 3D illustrates a cross-sectional view of an over-the-ear configuration of dual-fit earphones in accordance with the disclosed technology.

FIG. 3E illustrates a cross-sectional view of an over-the-ear configuration of the dual-fit earphones of FIG. 3D.

FIG. 3F illustrates a cross-sectional view of an under-the-ear configuration of the dual-fit earphones of FIG. 3D.

FIG. 4A is a block diagram illustrating an example computing device that may be used to implement embodiments of the disclosed technology.

FIG. 4B illustrates modules of an example activity monitoring application that may be used to implement embodiments of the disclosed technology.

FIG. 5 is an operational flow diagram illustrating a method of prompting a user to adjust the placement of earphones in the user's ear to ensure accurate biometric data collection by the earphones' biometric sensors.

FIG. 6A is an operational flow diagram illustrating an exemplary method for tracking biological age over time.

FIG. 6B is an operational flow diagram illustrating a particular embodiment of a method of calculating and displaying biological age over multiple time intervals.

FIG. 7A is a schematic block diagram illustrating one embodiment of a system for communicating biological age data between system modules.

FIG. 7B is a schematic block diagram illustrating a biological age calculation and display module.

FIG. 8 illustrates an activity display that may be associated with an activity display module of the activity monitoring application of FIG. 4B.

FIG. 9 illustrates a sleep display that may be associated with a sleep display module of the activity monitoring application of FIG. 4B.

FIG. 10 illustrates an activity recommendation and fatigue level display that may be associated with an activity recommendation and fatigue level display module of the activity monitoring application of FIG. 4B.

FIG. 11 illustrates a biological data and intensity recommendation display that may be associated with a biological data and intensity recommendation display module of the activity monitoring application of FIG. 4B.

FIG. 12 illustrates an example computing module that may be used to implement various features of the technology disclosed herein.

### DETAILED DESCRIPTION

Currently available technologies typically only measure biological age based on user input, and are not capable of tracking or monitoring biological age over time based on both user input and measured parameters. In particular, heart rate variability is a measurable parameter and is known to correlate to physiological resilience and behavioral flexibility, which are both important factors in determining biological age. However, currently available technologies do not leverage heart rate variability measurements, or other biological age determining factors measurable by activity monitoring devices, that could be used to semi-automate biological age tracking. In view of these drawbacks, there exists a long-felt need for fitness monitoring devices capable of combining user input with measured factors, including heart rate variability, to display and track changes in biological age over time.

The present disclosure is directed toward systems and methods for tracking biological age over time based. In particular embodiments, the systems and methods are directed to earphones with biometric sensors that are used to track biological age over time based upon heart rate variability.

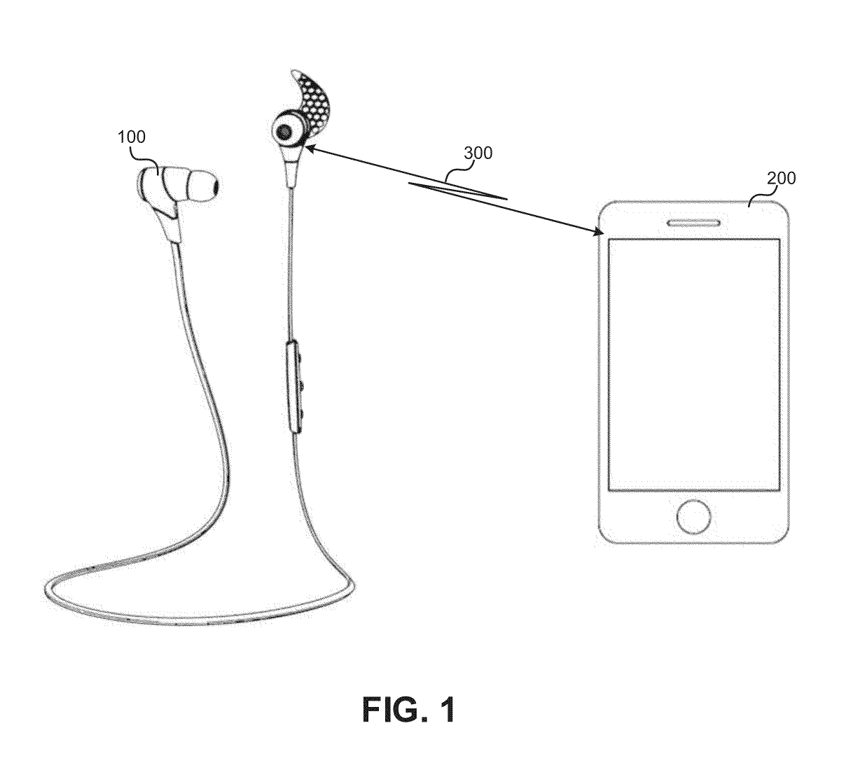

FIG. 1 illustrates an example communications environment in accordance with an embodiment of the technology disclosed herein. In this embodiment, earphones 100 communicate biometric and audio data with computing device 200 over a communication link 300. The biometric data is measured by one or more sensors (e.g., heart rate sensor, accelerometer, gyroscope) of earphones 100. Although a smartphone is illustrated, computing device 200 may comprise any computing device (smartphone, tablet, laptop, smartwatch, desktop, etc.) configured to transmit audio data to earphones 100, receive biometric data from earphones 100 (e.g., heartrate and motion data), and process the biometric data collected by earphones 100. In additional embodiments, computing device 200 itself may collect additional biometric information that is provided for display. For example, if computing device 200 is a smartphone it may use built in accelerometers, gyroscopes, and a GPS to collect additional biometric data.

Computing device 200 additionally includes a graphical user interface (GUI) to perform functions such as accepting user input and displaying processed biometric data to the user. The GUI may be provided by various operating systems known in the art, such as, for example, iOS, Android, Windows Mobile, Windows, Mac OS, Chrome OS, Linux, Unix, a gaming platform OS, etc. The biometric information displayed to the user can include, for example a summary of the user's activities, a summary of the user's fitness levels, activity recommendations for the day, the user's heart rate and heart rate variability (HRV), and other activity related information. User input that can be accepted on the GUI can include inputs for interacting with an activity tracking application further described below.

In preferred embodiments, the communication link 300 is a wireless communication link based on one or more wireless communication protocols such as BLUETOOTH, ZIGBEE, 802.11 protocols, Infrared (IR), Radio Frequency (RF), etc. Alternatively, the communications link 300 may be a wired link (e.g., using any one or a combination of an audio cable, a USB cable, etc.)

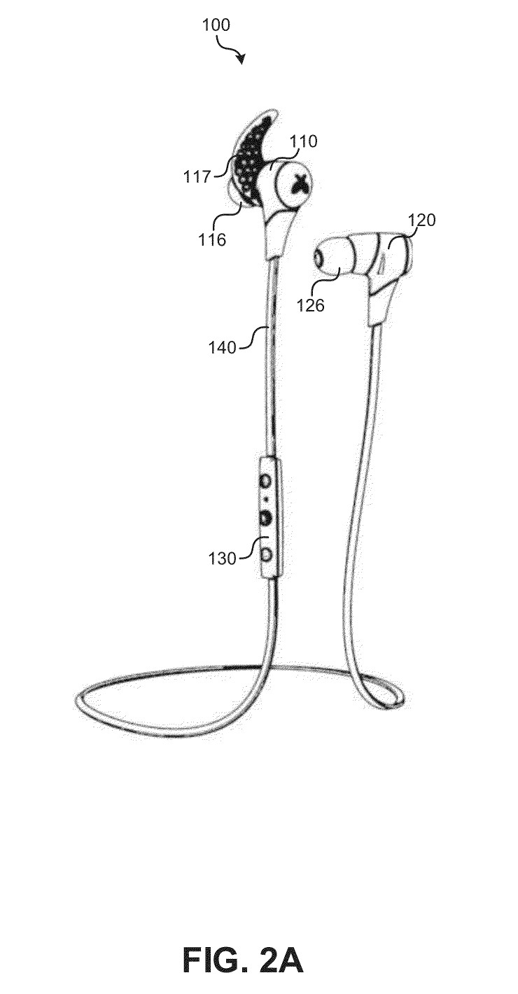

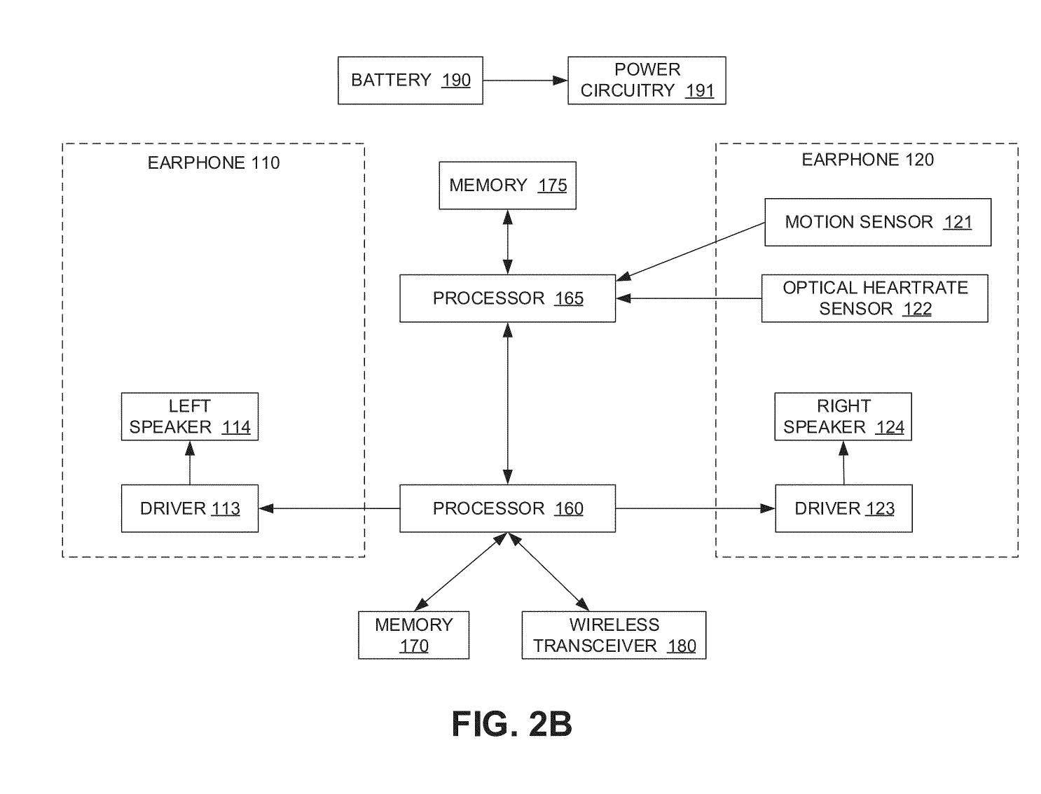

With specific reference now to earphones 100, FIG. 2A is a diagram illustrating a perspective view of exemplary earphones 100. FIG. 2A will be described in conjunction with FIG. 2B, which is a diagram illustrating an example architecture for circuitry of earphones 100. Earphones 100 comprise a left earphone 110 with tip 116, a right earphone 120 with tip 126, a controller 130 and a cable 140. Cable 140 electrically couples the right earphone 110 to the left earphone 120, and both earphones 110-120 to controller 130. Additionally, each earphone may optionally include a fin or ear cushion 117 that contacts folds in the outer ear anatomy to further secure the earphone to the wearer's ear.

In embodiments, earphones 100 may be constructed with different dimensions, including different diameters, widths, and thicknesses, in order to accommodate different human ear sizes and different preferences. In some embodiments of earphones 100, the housing of each earphone 110, 120 is rigid shell that surrounds electronic components. For example, the electronic components may include motion sensor 121, optical heartrate sensor 122, audio-electronic components such as drivers 113, 123 and speakers 114, 124, and other circuitry (e.g., processors 160, 165, and memories 170, 175). The rigid shell may be made with plastic, metal, rubber, or other materials known in the art. The housing may be cubic shaped, prism shaped, tubular shaped, cylindrical shaped, or otherwise shaped to house the electronic components.

The tips 116, 126 may be shaped to be rounded, parabolic, and/or semi-spherical, such that it comfortably and securely fits within a wearer's ear, with the distal end of the tip contacting an outer rim of the wearer's outer ear canal. In some embodiments, the tip may be removable such that it may be exchanged with alternate tips of varying dimensions, colors, or designs to accommodate a wearer's preference and/or fit more closely match the radial profile of the wearer's outer ear canal. The tip may be made with softer materials such as rubber, silicone, fabric, or other materials as would be appreciated by one of ordinary skill in the art.

In embodiments, controller 130 may provide various controls (e.g., buttons and switches) related to audio playback, such as, for example, volume adjustment, track skipping, audio track pausing, and the like. Additionally, controller 130 may include various controls related to biometric data gathering, such as, for example, controls for enabling or disabling heart rate and motion detection. In a particular embodiment, controller 130 may be a three button controller.

The circuitry of earphones 100 includes processors 160 and 165, memories 170 and 175, wireless transceiver 180, circuitry for earphone 110 and earphone 120, and a battery 190. In this embodiment, earphone 120 includes a motion sensor 121 (e.g., an accelerometer or gyroscope), an optical heartrate sensor 122, and a right speaker 124 and corresponding driver 123. Earphone 110 includes a left speaker 114 and corresponding driver 113. In additional embodiments, earphone 110 may also include a motion sensor (e.g., an accelerometer or gyroscope), and/or an optical heartrate sensor.

A biometric processor 165 comprises logical circuits dedicated to receiving, processing and storing biometric information collected by the biometric sensors of the earphones. More particularly, as illustrated in FIG. 2B, processor 165 is electrically coupled to motion sensor 121 and optical heartrate sensor 122, and receives and processes electrical signals generated by these sensors. These processed electrical signals represent biometric information such as the earphone wearer's motion and heartrate. Processor 165 may store the processed signals as biometric data in memory 175, which may be subsequently made available to a computing device using wireless transceiver 180. In some embodiments, sufficient memory is provided to store biometric data for transmission to a computing device for further processing.

During operation, optical heartrate sensor 122 uses a photoplethysmogram (PPG) to optically obtain the user's heart rate. In one embodiment, optical heartrate sensor 122 includes a pulse oximeter that detects blood oxygenation level changes as changes in coloration at the surface of a user's skin. More particularly, in this embodiment, the optical heartrate sensor 122 illuminates the skin of the user's ear with a light-emitting diode (LED). The light penetrates through the epidermal layers of the skin to underlying blood vessels. A portion of the light is absorbed and a portion is reflected back. The light reflected back through the skin of the user's ear is then obtained with a receiver (e.g., a photodiode) and used to determine changes in the user's blood oxygen saturation (SpO2) and pulse rate, thereby permitting calculation of the user's heart rate using algorithms known in the art (e.g., using processor 165). In this embodiment, the optical sensor may be positioned on one of the earphones such that it is proximal to the interior side of a user's tragus when the earphones are worn.

In various embodiments, optical heartrate sensor 122 may also be used to estimate a heart rate variable (HRV), i.e. the variation in time interval between consecutive heartbeats, of the user of earphones 100. For example, processor 165 may calculate the HRV using the data collected by sensor 122 based on a time domain methods, frequency domain methods, and other methods known in the art that calculate HRV based on data such as the mean heart rate, the change in pulse rate over a time interval, and other data used in the art to estimate HRV.

In further embodiments, logic circuits of processor 165 may further detect, calculate, and store metrics such as the amount of physical activity, sleep, or rest over a period of time, or the amount of time without physical activity over a period of time. The logic circuits may use the HRV, the metrics, or some combination thereof to calculate a recovery score. In various embodiments, the recovery score may indicate the user's physical condition and aptitude for further physical activity for the current day. For example, the logic circuits may detect the amount of physical activity and the amount of sleep a user experienced over the last 48 hours, combine those metrics with the user's HRV, and calculate a recovery score. In various embodiments, the calculated recovery score may be based on any scale or range, such as, for example, a range between 1 and 10, a range between 1 and 100, or a range between 0% and 100%.

During audio playback, earphones 100 wirelessly receive audio data using wireless transceiver 180. The audio data is processed by logic circuits of audio processor 160 into electrical signals that are delivered to respective drivers 113 and 123 of left speaker 114 and right speaker 124 of earphones 110 and 120. The electrical signals are then converted to sound using the drivers. Any driver technologies known in the art or later developed may be used. For example, moving coil drivers, electrostatic drivers, electret drivers, orthodynamic drivers, and other transducer technologies may be used to generate playback sound.

The wireless transceiver 180 is configured to communicate biometric and audio data using available wireless communications standards. For example, in some embodiments, the wireless transceiver 180 may be a BLUETOOTH transmitter, a ZIGBEE transmitter, a Wi-Fi transmitter, a GPS transmitter, a cellular transmitter, or some combination thereof. Although FIG. 2B illustrates a single wireless transceiver 180 for both transmitting biometric data and receiving audio data, in an alternative embodiment, a transmitter dedicated to transmitting only biometric data to a computing device may be used. In this alternative embodiment, the transmitter may be a low energy transmitter such as a near field communications (NFC) transmitter or a BLUETOOTH low energy (LE) transmitter. In implementations of this particular embodiment, a separate wireless receiver may be provided for receiving high fidelity audio data from an audio source. In yet additional embodiments, a wired interface (e.g., micro-USB) may be used for communicating data stored in memories 165 and 175.

FIG. 2B also shows that the electrical components of headphones 100 are powered by a battery 190 coupled to power circuity 191. Any suitable battery or power supply technologies known in the art or later developed may be used. For example, a lithium-ion battery, aluminum-ion battery, piezo or vibration energy harvesters, photovoltaic cells, or other like devices can be used. In embodiments, battery 190 may be enclosed in earphone 110 or earphone 120. Alternatively, battery 102 may be enclosed in controller 130. In embodiments, the circuitry may be configured to enter a low-power or inactive mode when earphones 100 are not in use. For example, mechanisms such as, for example, an on/off switch, a BLUETOOTH transmission disabling button, or the like may be provided on controller 130 such that a user may manually control the on/off state of power-consuming components of earphones 100.

It should be noted that in various embodiments, processors 160 and 165, memories 170 and 175, wireless transceiver 180, and battery 190 may be enclosed in and distributed throughout any one or more of earphone 110, earphone 120, and controller 130. For example, in one particular embodiment, processor 165 and memory 175 may be enclosed in earphone 120 along with optical heartrate sensor 122 and motion sensor 121. In this particular embodiment, these four components are electrically coupled to the same printed circuit board (PCB) enclosed in earphone 120. It should also be noted that although audio processor 160 and biometric processor 165 are illustrated in this exemplary embodiment as separate processors, in an alternative embodiment the functions of the two processors may be integrated into a single processor.

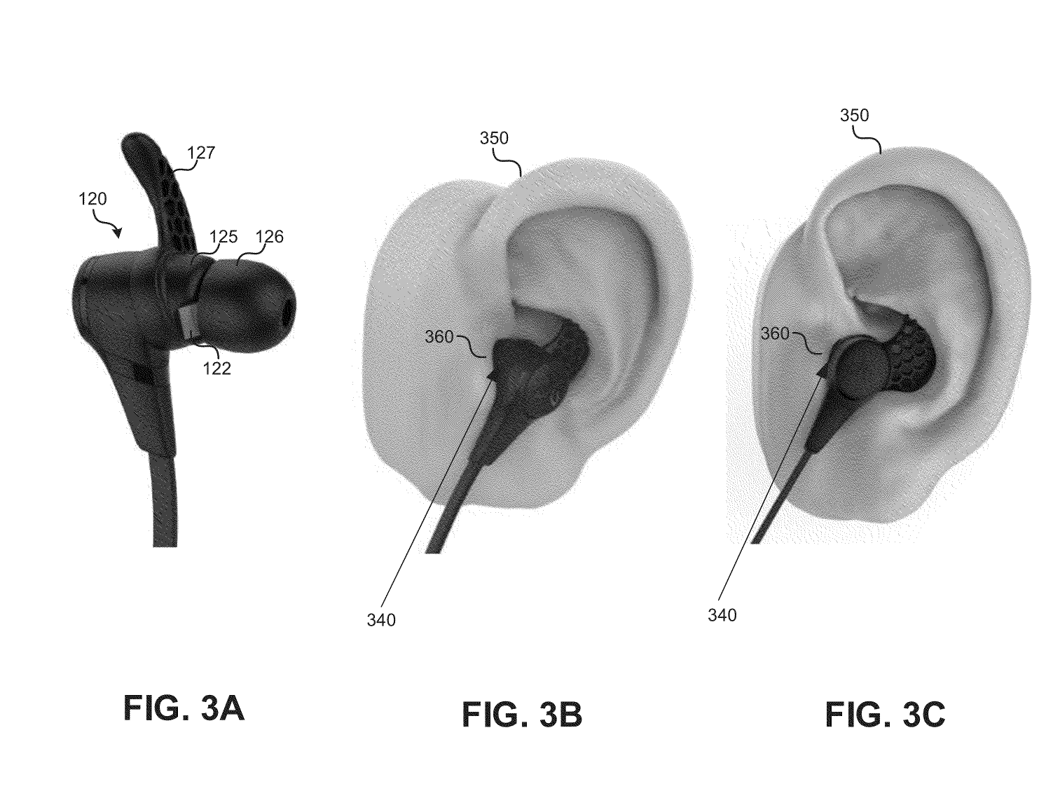

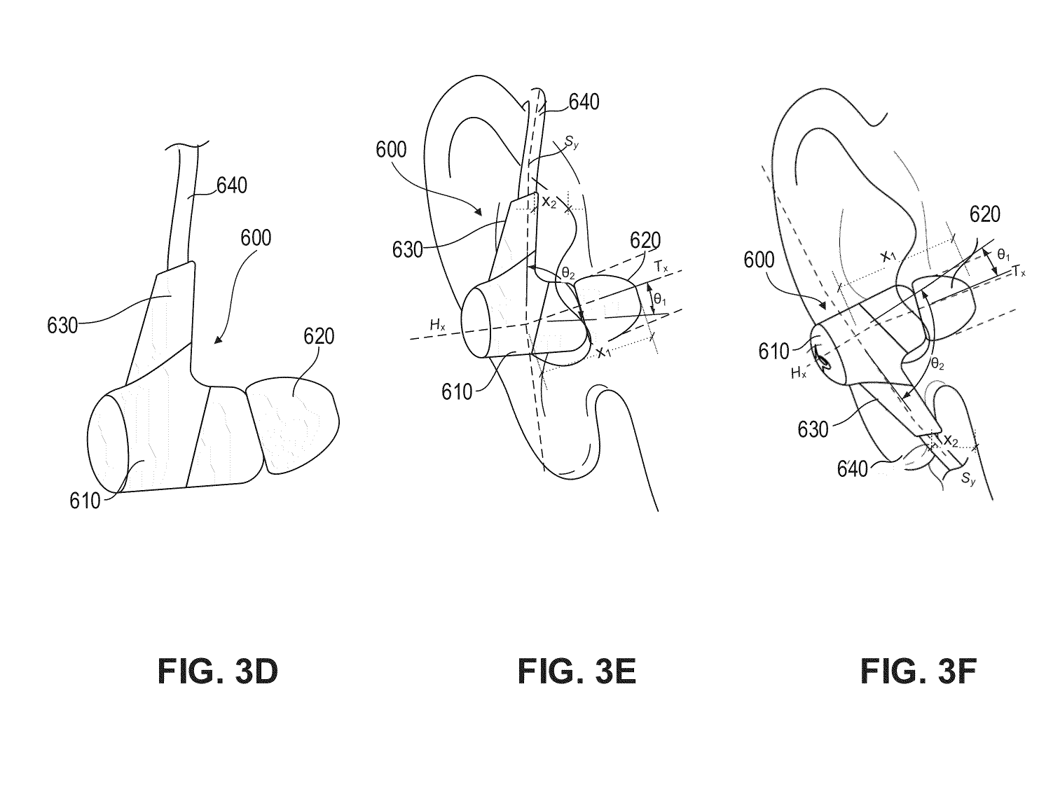

FIG. 3A illustrates a perspective view of one embodiment of an earphone 120, including an optical heartrate sensor 122, in accordance with the technology disclosed herein. FIG. 3A will be described in conjunction with FIGS. 3B-3C, which are perspective views illustrating placement of heartrate sensor 122 when earphone 120 is worn in a user's ear 350. As illustrated, earphone 120 includes a body 125, tip 126, ear cushion 127, and an optical heartrate sensor 122. Optical heartrate sensor 122 protrudes from a frontal side of body 125, proximal to tip 126 and where the earphone's nozzle (not shown) is present. FIGS. 3B-3C illustrate the optical sensor and ear interface 340 when earphone 120 is worn in a user's ear 350. When earphone 120 is worn, optical heartrate sensor 122 is proximal to the interior side of a user's tragus 360.

In this embodiment, optical heartrate sensor 122 illuminates the skin of the interior side of the ear's tragus 360 with a light-emitting diode (LED). The light penetrates through the epidermal layers of the skin to underlying blood vessels. A portion of the light is absorbed and a portion is reflected back. The light reflected back through the skin is then obtained with a receiver (e.g., a photodiode) of optical heartrate sensor 122 and used to determine changes in the user's blood flow, thereby permitting measurement of the user's heart rate and HRV.

In various embodiments, earphones 100 may be dual-fit earphones shaped to comfortably and securely be worn in either an over-the-ear configuration or an under-the-ear configuration. The secure fit provided by such embodiments keeps the optical heartrate sensor 122 in place on the interior side of the ear's tragus 360, thereby ensuring accurate and consistent measurements of a user's heartrate.

FIGS. 3D and 3E are cross-sectional views illustrating one such embodiment of dual-fit earphones 600 being worn in an over-the-ear configuration. FIG. 3F illustrates dual-fit earphones 600 in an under-the-ear configuration.

As illustrated, earphone 600 includes housing 610, tip 620, strain relief 630, and cord or cable 640. The proximal end of tip 620 mechanically couples to the distal end of housing 610. Similarly, the distal end of strain relief 630 mechanically couples to a side (e.g., the top side) of housing 610. Furthermore, the distal end of cord 640 is disposed within and secured by the proximal end of strain relief 630. The longitudinal axis of the housing, Hₓ, forms angle θ₁ with respect to the longitudinal axis of the tip, Tₓ. The longitudinal axis of the strain relief, S_{y}, aligns with the proximal end of strain relief 630 and forms angle θ₂ with respect to the axis Hₓ. In several embodiments, θ₁ is greater than 0 degrees (e.g., Tₓ extends in a non-straight angle from Hₓ, or in other words, the tip 620 is angled with respect to the housing 610). In some embodiments, θ₁ is selected to approximate the ear canal angle of the wearer. For example, θ₁ may range between 5 degrees and 15 degrees. Also in several embodiments, θ₂ is less than 90 degrees (e.g., S_{y} extends in a non-orthogonal angle from Hₓ, or in other words, the strain relief 630 is angled with respect to a perpendicular orientation with housing 610). In some embodiments, θ₂ may be selected to direct the distal end of cord 640 closer to the wearer's ear. For example, θ₂ may range between 75 degrees and 89 degrees.

As illustrated, x₁ represents the distance between the distal end of tip 620 and the intersection of strain relief longitudinal axis S_{y} and housing longitudinal axis Hₓ. One of skill in the art would appreciate that the dimension x₁ may be selected based on several parameters, including the desired fit to a wearer's ear based on the average human ear anatomical dimensions, the types and dimensions of electronic components (e.g., optical sensor, motion sensor, processor, memory, etc.) that must be disposed within the housing and the tip, and the specific placement of the optical sensor. In some examples, x₁ may be at least 18 mm. However, in other examples, x₁ may be smaller or greater based on the parameters discussed above.

Similarly, as illustrated, x₂ represents the distance between the proximal end of strain relief 630 and the surface wearer's ear. In the configuration illustrated, θ₂ may be selected to reduce x₂, as well as to direct the cord 640 towards the wearer's ear, such that cord 640 may rest in the crevice formed where the top of the wearer's ear meets the side of the wearer's head. In some embodiments, θ₂ may range between 75 degrees and 85 degrees. In some examples, strain relief 630 may be made of a flexible material such as rubber, silicone, or soft plastic such that it may be further bent towards the wearer's ear. Similarly, strain relief 630 may comprise a shape memory material such that it may be bent inward and retain the shape. In some examples, strain relief 630 may be shaped to curve inward towards the wearer's ear.

In some embodiments, the proximal end of tip 620 may flexibly couple to the distal end of housing 610, enabling a wearer to adjust θ₁ to most closely accommodate the fit of tip 620 into the wearer's ear canal (e.g., by closely matching the ear canal angle).

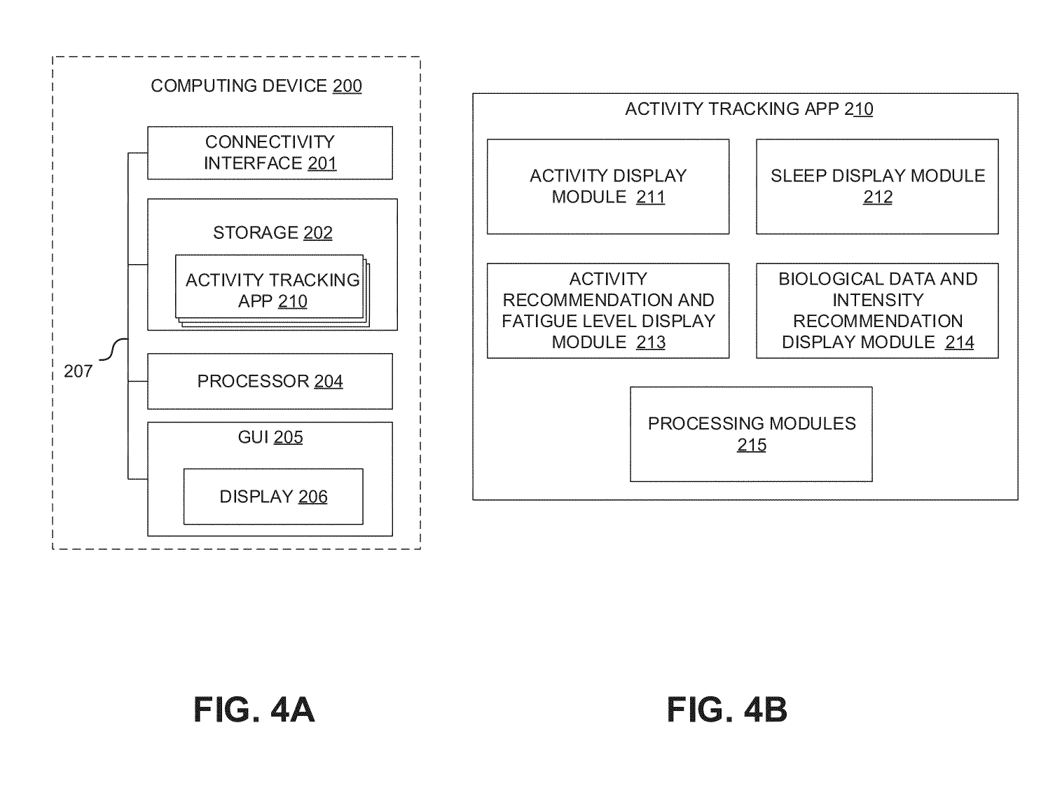

As one having skill in the art would appreciate from the above description, earphones 100 in various embodiments may gather biometric user data that may be used to track a user's activities and activity level. That data may then be made available to a computing device, which may provide a GUI for interacting with the data using a software activity tracking application installed on the computing device. FIG. 4A is a block diagram illustrating example components of one such computing device 200 including an installed activity tracking application 210.

As illustrated in this example, computing device 200 comprises a connectivity interface 201, storage 202 with activity tracking application 210, processor 204, a graphical user interface (GUI) 205 including display 206, and a bus 207 for transferring data between the various components of computing device 200.

Connectivity interface 201 connects computing device 200 to earphones 100 through a communication medium. The medium may comprise a wireless network system such as a BLUETOOTH system, a ZIGBEE system, an Infrared (IR) system, a Radio Frequency (RF) system, a cellular network, a satellite network, a wireless local area network, or the like. The medium may additionally comprise a wired component such as a USB system.

Storage 202 may comprise volatile memory (e.g. RAM), non-volatile memory (e.g. flash storage), or some combination thereof. In various embodiments, storage 202 may store biometric data collected by earphones 100. Additionally, storage 202 stores an activity tracking application 210, that when executed by processor 204, allows a user to interact with the collected biometric information.

In various embodiments, a user may interact with activity tracking application 210 via a GUI 205 including a display 206, such as, for example, a touchscreen display that accepts various hand gestures as inputs. In accordance with various embodiments, activity tracking application 210 may process the biometric information collected by earphones 100 and present it via display 206 of GUI 205. Before describing activity tracking application 210 in further detail, it is worth noting that in some embodiments earphones 100 may filter the collected biometric information prior to transmitting the biometric information to computing device 200. Accordingly, although the embodiments disclosed herein are described with reference to activity tracking application 210 processing the received biometric information, in various implementations various preprocessing operations may be performed by a processor 160, 165 of earphones 100.

In various embodiments, activity tracking application 210 may be initially configured/setup (e.g., after installation on a smartphone) based on a user's self-reported biological information, sleep information, and activity preference information. For example, during setup a user may be prompted via display 206 for biological information such as the user's gender, height, age, and weight. Further, during setup the user may be prompted for sleep information such as the amount of sleep needed by the user and the user's regular bed time. Further, still, the user may be prompted during setup for a preferred activity level and activities the user desires to be tracked (e.g., running, walking, swimming, biking, etc.) In various embodiments, described below, this self-reported information may be used in tandem with the information collected by earphones 100 to display activity monitoring information using various modules.

Following setup, activity tracking application 210 may be used by a user to monitor and define how active the user wants to be on a day-to-day basis based on the biometric information (e.g., accelerometer information, optical heart rate sensor information, etc.) collected by earphones 100. As illustrated in FIG. 4B, activity tracking application 210 may comprise various display modules, including an activity display module 211, a sleep display module 212, an activity recommendation and fatigue level display module 213, and a biological data and intensity recommendation display module 214. Additionally, activity tracking application 210 may comprise various processing modules 215 for processing the activity monitoring information (e.g., optical heartrate information, accelerometer information, gyroscope information, etc.) collected by the earphones or the biological information entered by the users. These modules may be implemented separately or in combination. For example, in some embodiments activity processing modules 215 may be directly integrated with one or more of display modules 211-214.

As will be further described below, each of display modules 211-214 may be associated with a unique display provided by activity tracking app 210 via display 206. That is, activity display module 211 may have an associated activity display, sleep display module 212 may have an associated sleep display, activity recommendation and fatigue level display module 213 may have an associated activity recommendation and fatigue level display, and biological data and intensity recommendation display module 214 may have an associated biological data and intensity recommendation display.

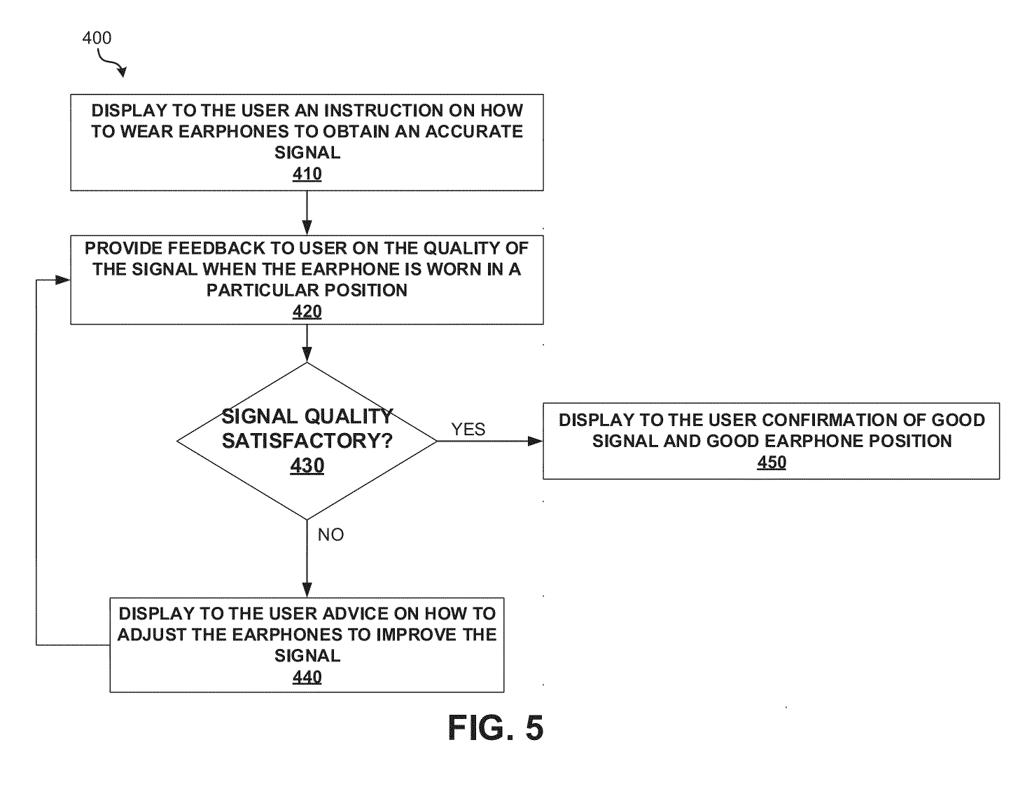

In embodiments, application 210 may be used to display to the user an instruction for wearing and/or adjusting earphones 100 if it is determined that optical heartrate sensor 122 and/or motion sensor 121 are not accurately gathering motion data and heart rate data. FIG. 5 is an operational flow diagram illustrating one such method 400 of an earphone adjustment feedback loop with a user that ensures accurate biometric data collection by earphones 100. At operation 410, execution of application 210 may cause display 206 to display an instruction to the user on how to wear earphones 100 to obtain an accurate and reliable signal from the biometric sensors. In embodiments, operation 410 may occur once after installing application 210, once a day (e.g., when user first wears the earphones 100 for the day), or at any customizable and/or predetermined interval.

At operation 420, feedback is displayed to the user regarding the quality of the signal received from the biometric sensors based on the particular position that earphones 100 are being worn. For example, display 206 may display a signal quality bar or other graphical element. At decision 430, it is determined if the biosensor signal quality is satisfactory for biometric data gathering and use of application 210. In various embodiments, this determination may be based on factors such as, for example, the frequency with which optical heartrate sensor 122 is collecting heart rate data, the variance in the measurements of optical heartrate sensor 122, dropouts in heart rate measurements by sensor 122, the signal-to-noise ratio approximation of optical heartrate sensor 122, the amplitude of the signals generated by the sensors, and the like.

If the signal quality is unsatisfactory, at operation 440, application 210 may cause display 206 to display to the user advice on how to adjust the earphones to improve the signal, and operations 420 and decision 430 may subsequently be repeated. For example, advice on adjusting the strain relief of the earphones may be displayed. Otherwise, if the signal quality is satisfactory, at operation 450, application may cause display 206 to display to the user confirmation of good signal quality and/or good earphone position. Subsequently, application 210 may proceed with normal operation (e.g., display modules 211-214).

In various embodiments, earphones 100 and computing device 200 may be implemented in a method and system for tracking biological age over time.

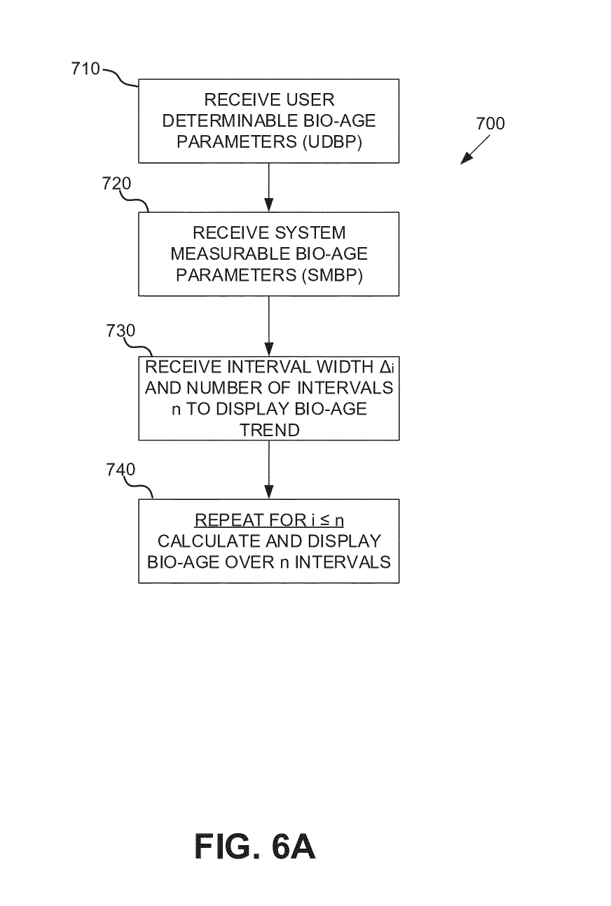

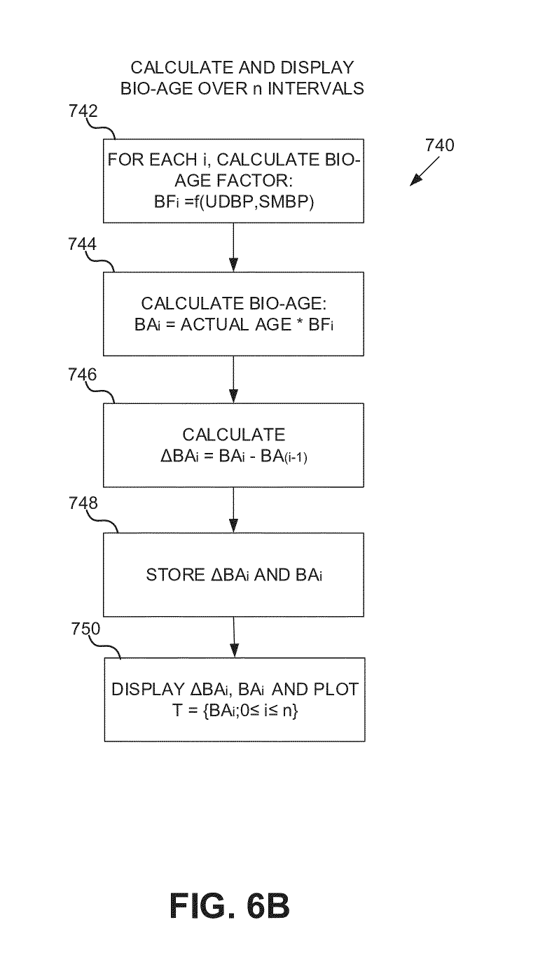

FIG. 6A is an operational flow diagram illustrating an exemplary method 700 for tracking biological age over time. Method 700 includes receiving one or more user determinable biological age parameters (UDBP) at operation 710, receiving one or more system measurable biological age parameters (SMBP) 720, receiving an interval width (Δᵢ) and number of intervals (n) at operation 730, and calculating a biological age (BA) for each i between 0 and n at operation 740.

FIG. 6B is an operational flow diagram illustrating a particular embodiment of a method or operation 740 of calculating and displaying biological age over multiple time intervals. As illustrated by FIG. 6B, method 740 includes (i) calculating a biological age factor (BFᵢ) as a function of each UDBP and each SMBP at operation 742, as illustrated by Equation 1; (ii) calculating BAᵢ as the product of BFᵢ and a user's actual age at operation 744, as illustrated by Equation 2; (iii) calculating a change in biological age between intervals at operation 746, as illustrated by Equation 3; (iv) storing ΔBAᵢ and BAᵢ at operation 748; and (v) displaying ΔBAᵢ, BAᵢ, and a biological age trend at operation 750, as illustrated by the trend T in Equation 4. In various embodiments, the displaying at step 750 may be performed by a mobile device or other computing device 200 (e.g., using activity tracking application 210).

BFᵢ=f(UDBP,SMBP) (1)

BAᵢ=Age*BFᵢ (2)

ΔBAᵢ=BAᵢ−BAᵢ₋₁ (3)

T={BAᵢ;0≦i≦n} (4)

UDBP=ΠUDBPⱼ (5)

SMBP=ΠSMBPₖ (6)

Referring to Equation 1, UDBP is a product function of factors that may include a user's actual age, gender, ethnicity, eating habits, stress level profile related to job or personal life, marriage profile, height, weight, body fat index, health metrics such as cholesterol level, blood pressure, and medical history, and other factors known to affect longevity of human life. In various embodiments, these factors may entered by the user (e.g., using application 210), obtained from storage in a local computing device, and/or obtained over a network from a server. Each factor, UDBPⱼ, may be given a numerical weighting indicating its effect on either extending or reducing average life based on statistical patterns that are well known in the art. Because the UDBPⱼ are combined in a product function, as illustrated by Equation 5, each UDBPⱼ with neutral effect on human longevity may have a value equal to 1, each life-extending UDBPⱼ may have a value greater than 1, and life-reducing UDBPⱼ may have a value of less than 1. For example, women tend to live longer than men, and therefore, a male UDBPⱼ would have a value of less than 1 and a female UDBPⱼ would have a value of greater than 1. Health conditions that are known to dramatically reduce life may have a UDBPⱼ value of much less than 1. Other factors may have UDBPⱼ values of less than 1 or more than 1 as scaled by the level that the factor affects human longevity. This data may be readily pulled from publicly available sources. Each UDBPⱼ may be combined in a product function, as shown in Equation 5, to result in a combined UDBP factor. Some UDBPⱼ may change over time, such as a user's actual age, marriage profile, stress profile, or health profile. Accordingly, UDBP is not constant across biological age calculation intervals. Other UDBPⱼ factors may be incorporated into the biological age factor calculation in Equation 1 as would be known to one of ordinary skill in the art.

Still referring to Equation 1, SMBP is a product function of factors that may be measured and/or calculated by the activity monitoring device, or entered by the user. SMBP factors, SMBPₖ, may include sleep profile, activity profile, recovery scores based on heart rate variability, activity scores, and smart activity scores. Just as with UDBP, because the SMBPₖ are combined in a product function, as illustrated in Equation 6, each SMBPₖ with neutral effect on human longevity may have a value equal to 1, each life-extending SMBPₖ may have a value greater than 1, and each life-reducing SMBPₖ may have a value of less than 1. For example, users who do not sleep or regularly participate in activity may have sleep or activity profile with SMBPₖ values of less than 1, whereas users with higher activity scores may have activity score SMBPₖ values of greater than 1. Further, users with more resilient HRVs may have HRV SMBPₖ values of greater than 1, whereas users with less resilient HRVs may have HRV SMBPₖ values of less than 1. As discussed, each SMBPₖ may be combined in a product function, as illustrated in Equation 6, to generate a combined SMBP factor. Each SMBPₖ may change over time depending on changes in measured results from the activity monitoring device or entries from the user. Other SMBPₖ may be incorporated into the biological age factor calculation in Equation 1 as would be known to one of ordinary skill in the art.

Still referring to Equation 1, the sleep profile SMBPₖ may refer to both the quality and quantity of sleep. For example, the sleep quality metric may represent the percentage of a user's sleep that is uninterrupted, and the sleep quantity metric may represent the user's total hours per day of sleep compared to average hours of sleep per day across a particular population or as compared to recommended hours of sleep. Similarly, the activity profile SMBPₖ may refer to both quality and quantity of activity. For example, the activity quality metric may represent the average intensity level of a user's activity, and the activity quantity metric may represent the total hours per day of activity as compared with an average total hours per day of activity across a population or as compared to a recommended total hours of activity.

In various embodiments, the sleep profile, activity profile, activity score, and smart activity score SMBPₖ may be determined by processing electric signals received from sensors of earphones 100 and/or computing device 200. For example, in one particular embodiment, the electrical signals generated by motion sensor 121 and heartrate sensor 122 of earphones 100 may be used to track a user's motions and corresponding heart rate and/or HRV to determine the user's sleep profile, activity profile, activity score, and smart activity score. Particular embodiments of implementing this functionality are described in greater detail U.S. patent application Ser. No. 14/568,835, filed Dec. 12, 2014, titled “System and Method for Creating a Dynamic Activity Profile”, and U.S. patent application Ser. No. 14/137,734, filed Dec. 20, 2013, titled “System and Method for Providing a Smart Activity Score”, both of which are incorporated herein by reference in their entirety.

Referring to Equation 2, UDBP and SMBP may be combined in a single biological age factor (BFᵢ) and multiplied by actual age to generate a biological age (BAᵢ) for any given time interval i. As both UDBP and SMBP values may change over time, the BFᵢ may also change over time, and consequently, the BAᵢ may also change over time.

Referring to Equation 3, biological age calculations BAᵢ from different each time interval i may be compared with the biological age calculation BAᵢ₋₁ from the immediately preceding time interval (i−1) to generate a ΔBAᵢ.

Referring to Equation 4, a biological age trend function y(BA) may plot each BAᵢ against each i from an initial time interval i=0 to a current time interval i=n.

In some embodiments, a method for tracking and displaying biological age may include calculating and displaying recommendations to the user (e.g., using application 210). Some of these embodiments may include systems and methods for comparing biological age with physical activity monitoring, calculating recommended activity regiments to reduce, maintain, or achieve a desired biological age, and monitoring progress towards achieving the desired biological age. For example, the user may determine a target biological age and adjust specific UDBPⱼ and/or SMBPₖ to achieve that target. Alternatively, one embodiment may include calculating adjustments to UDBPⱼ and/or SMBPₖ required to match the user's biological age to the user's actual age. In order to decrease biological age, the user may be required to increase sleep and/or activity quantity or quality, eat healthier, or find ways to lower stress. These adjustments may be monitored and progress tracked by displaying the biological age trend function plot as described in Equation 4 and at step 750.

In some examples, a system and method for tracking biological age over time may also displays physical activity trends over time, and may additionally flag changes in biological age on a correlated or combined display with physical activity trend changes such that a user may determine what physical activity characteristics may have caused the change in biological age. In one example, the system may also analyze the correlation between changes in biological age and lifestyle (e.g. physical activity, sleep patterns, eating habits, etc.) by using historical or known data trends to predict display advise indicating what lifestyle conditions may have affected the biological age change(s). In this example, a lifestyle trend may be displayed in a temporally synchronous display with a biological age trend. Here, temporally synchronous means that the lifestyle trend and biological age trend share the same temporal axis such that corresponding points on the lifestyle trend and biological trend are each derived from data collected at the same time. The system may further analyze, predict, and display advice indicating possible lifestyle changes would be necessary to return the biological age to a target value.

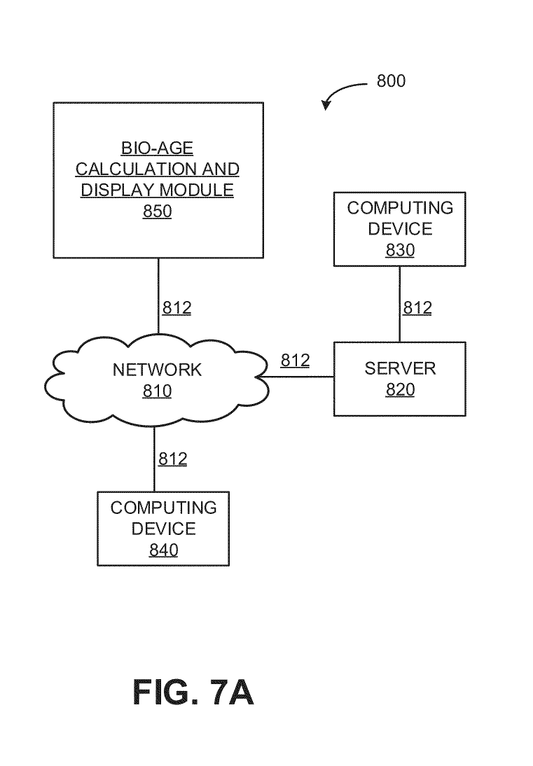

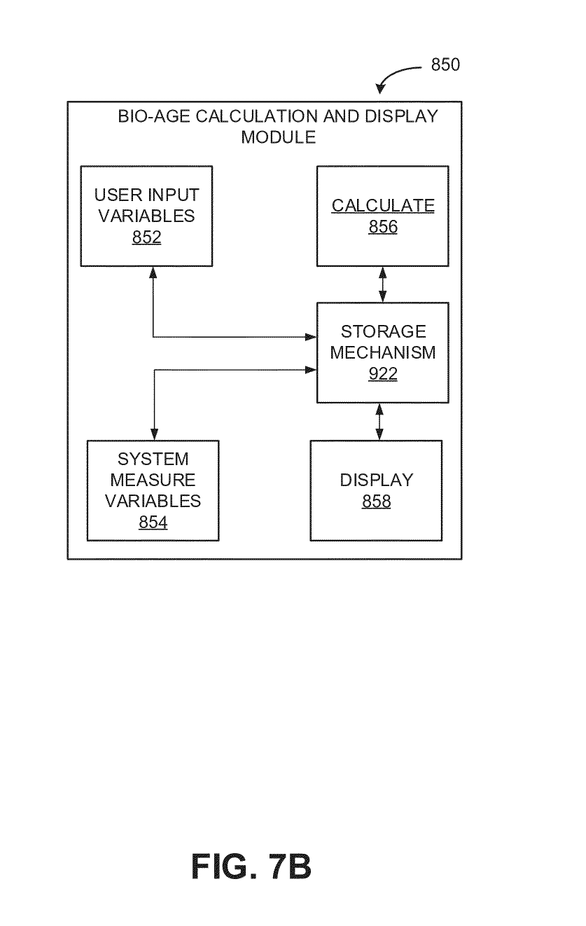

FIG. 7A is a schematic block diagram illustrating one embodiment of a system for communicating biological age data between system modules. System 800 includes multiple devices for communicating, calculating, and displaying biological age. For example, biological age calculation and display module 850 may connect to network 810 via communications mechanisms 812, 814, 816, and 818. The communications mechanisms may include various known technologies, including WAN, LAN, Wi-FI, TCP/IP, Bluetooth®, 4G LTE, or other known communications standards. The system for communicating metrics of interest may also include server 820 and computing devices 830 and 840.

Communication network 810 may be implemented in a variety of forms. For example, communication network 810 may be an Internet connection, such as a local area network (“LAN”), a wide area network (“WAN”), a fiber optic network, internet over power lines, a hard-wired connection (e.g., a bus), and the like, or any other kind of network connection. Communication network 804 may be implemented using any combination of routers, cables, modems, switches, fiber optics, wires, radio, and the like and using various wireless standards, such as Bluetooth®, Wi-FI, or 4G LTE such as to be compatible with the communications mechanisms.

Server 820 may direct communications made over communications network 810. Server 820 may be, for example, an Internet server, a router, a desktop or laptop computer, a smartphone, a tablet, a processor, a module, or the like. In one embodiment, server 820 directs communications between communications network 810 and computing devices 830 and/or 840. For example, server 820 may update information stored on computing device 830, or server 820 may send information to computing device 840 in real time.

Computing device 840 may take a variety of forms, such as a desktop or laptop computer, a smartphone, a tablet, a processor, a module, or the like. In addition, computing device 840 may be a module, processor, and/or other electronics embedded in a wearable device such as earphones, a bracelet, a smartwatch, a piece of clothing, and so forth. For example, computing device 840 may be substantially similar to electronics embedded in earphones 100. Computing device 840 may communicate with other devices over communication network 810 with or without the use of server 820. In one embodiment, computing device 840 includes earphones 100.

FIG. 7B is a schematic block diagram illustrating a biological age calculation and display module 850. Biological age calculation and display module 850 may comprise user determinable biological parameter (UDBP) receiving module 852, system measurable biological parameter (SMBP) receiving module 854, biological factor (BF) calculation module 856, display module 858, and storage mechanism 922. The UDBP receiving module 852 may be configured to receive input of UDBPⱼ from users. User input into the UDBP module may be stored in storage mechanism 922. The SMBP receiving module 854 may be configured to receive input of SMBPₖ from users or from an activity monitoring device. For example, earphones 100 may detect and calculate SMBPₖ and transmit them to SMBP receiving module 854. User input into the SMBP receiving module may be stored in storage mechanism 922. BF calculation module 856 may receive UDBPⱼ and SMBPₖ from storage mechanism 922 and calculate biological age factors, biological age, biological age changes over time, and a biological age trend function as shown in Equation 4. Display module 858 may be configured to display biological age, changes in biological age, and a biological age trend plot (e.g., using computing device 200).

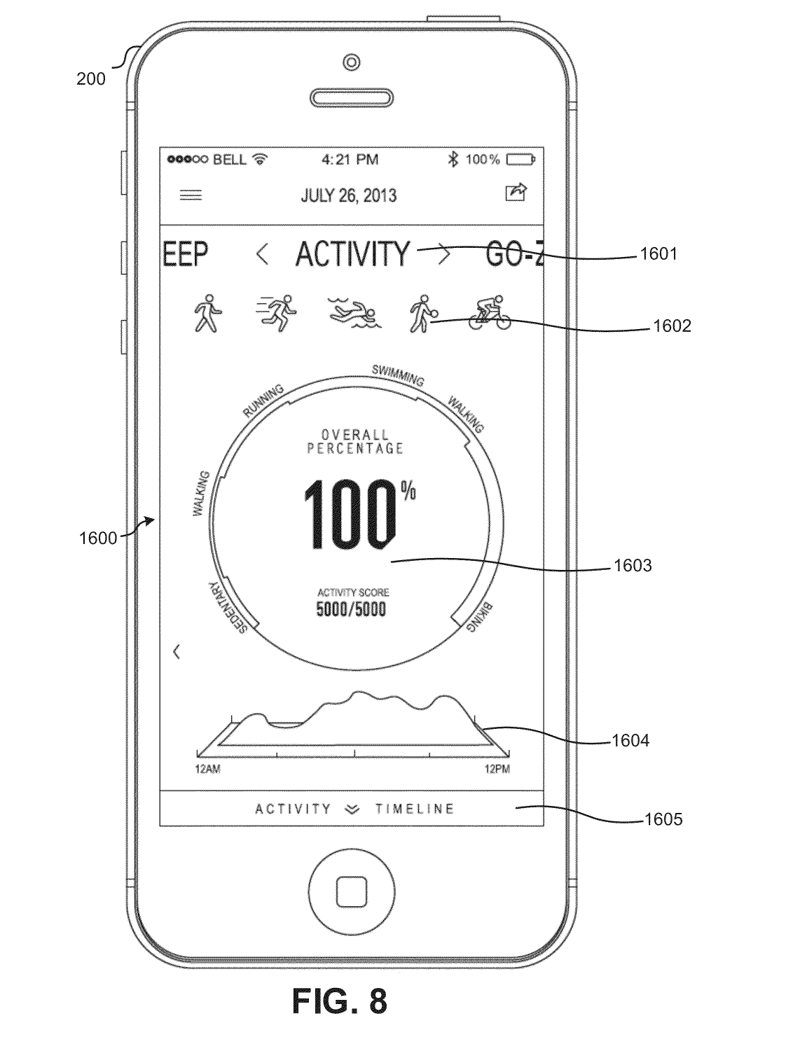

FIGS. 8-11 illustrate a particular implementation of a GUI for activity tracking application 210 comprising displays associated with each of display modules 211-214. In various embodiments, the GUI of activity tracking application 210 may be used by a user to track the user's biological age over time.

FIG. 8 illustrates an activity display 1600 that may be associated with an activity display module 211. In various embodiments, activity display 1600 may visually present to a user a record of the user's activity. As illustrated, activity display 1600 may comprise a display navigation area 1601, activity icons 1602, activity goal section 1603, live activity chart 1604, and activity timeline 1605. As illustrated in this particular embodiment, display navigation area 1601 allows a user to navigate between the various displays associated with modules 211-214 by selecting “right” and “left” arrows depicted at the top of the display on either side of the display screen title. An identification of the selected display may be displayed at the center of the navigation area 1601. Other selectable displays may displayed on the left and right sides of navigation area 1601. For example, in this embodiment the activity display 1600 includes the identification “ACTIVITY” at the center of the navigation area. If the user wishes to navigate to a sleep display in this embodiment, the user may select the left arrow. In implementations where device 200 includes a touch screen display, navigation between the displays may be accomplished via finger swiping gestures. For example, in one embodiment a user may swipe the screen right or left to navigate to a different display screen. In another embodiment, a user may press the left or right arrows to navigate between the various display screens.

In various embodiments, activity icons 1602 may be displayed on activity display 1600 based on the user's predicted or self-reported activity. For example, in this particular embodiment activity icons 1602 are displayed for the activities of walking, running, swimming, sport, and biking, indicating that the user has performed these five activities. In one particular embodiment, one or more modules of application 210 may estimate the activity being performed (e.g., sleeping, walking, running, or swimming) by comparing the data collected by a biometric earphone's sensors to pre-loaded or learned activity profiles. For example, accelerometer data, gyroscope data, heartrate data, or some combination thereof may be compared to preloaded activity profiles of what the data should look like for a generic user that is running, walking, or swimming. In implementations of this embodiment, the preloaded activity profiles for each particular activity (e.g., sleeping, running, walking, or swimming) may be adjusted over time based on a history of the user's activity, thereby improving the activity predictive capability of the system. In additional implementations, activity display 1600 allows a user to manually select the activity being performed (e.g., via touch gestures), thereby enabling the system to accurately adjust an activity profile associated with the user-selected activity. In this way, the system's activity estimating capabilities will improve over time as the system learns how particular activity profiles match an individual user. Particular methods of implementing this activity estimation and activity profile learning capability are described in U.S. patent application Ser. No. 14/568,835, filed Dec. 12, 2014, titled “System and Method for Creating a Dynamic Activity Profile”, and which is incorporated herein by reference in its entirety.

In various embodiments, an activity goal section 1603 may display various activity metrics such as a percentage activity goal providing an overview of the status of an activity goal for a timeframe (e.g., day or week), an activity score or other smart activity score associated with the goal, and activities for the measured timeframe (e.g., day or week). For example, the display may provide a user with a current activity score for the day versus a target activity score for the day. Particular methods of calculating activity scores are described in U.S. patent application Ser. No. 14/137,734, filed Dec. 20, 2013, titled “System and Method for Providing a Smart Activity Score”, and which is incorporated herein by reference in its entirety.

In various embodiments, the percentage activity goal may be selected by the user (e.g., by a touch tap) to display to the user an amount of a particular activity (e.g., walking or running) needed to complete the activity goal (e.g., reach 100%). In additional embodiments, activities for the timeframe may be individually selected to display metrics of the selected activity such as points, calories, duration, or some combination thereof. For example, in this particular embodiment activity goal section 1603 displays that 100% of the activity goal for the day has been accomplished. Further, activity goal section 1603 displays that activities of walking, running, biking, and no activity (sedentary) were performed during the day. This is also displayed as a numerical activity score 5000/5000. In this embodiment, a breakdown of metrics for each activity (e.g., activity points, calories, and duration) for the day may be displayed by selecting the activity.

A live activity chart 1604 may also display an activity trend of the aforementioned metrics (or other metrics) as a dynamic graph at the bottom of the display. For example, the graph may be used to show when user has been most active during the day (e.g., burning the most calories or otherwise engaged in an activity).

An activity timeline 1605 may be displayed as a collapsed bar at the bottom of display 1600. In various embodiments, when a user selects activity timeline 1605, it may display a more detailed breakdown of daily activity, including, for example, an activity performed at a particular time with associated metrics, total active time for the measuring period, total inactive time for the measuring period, total calories burned for the measuring period, total distance traversed for the measuring period, and other metrics.

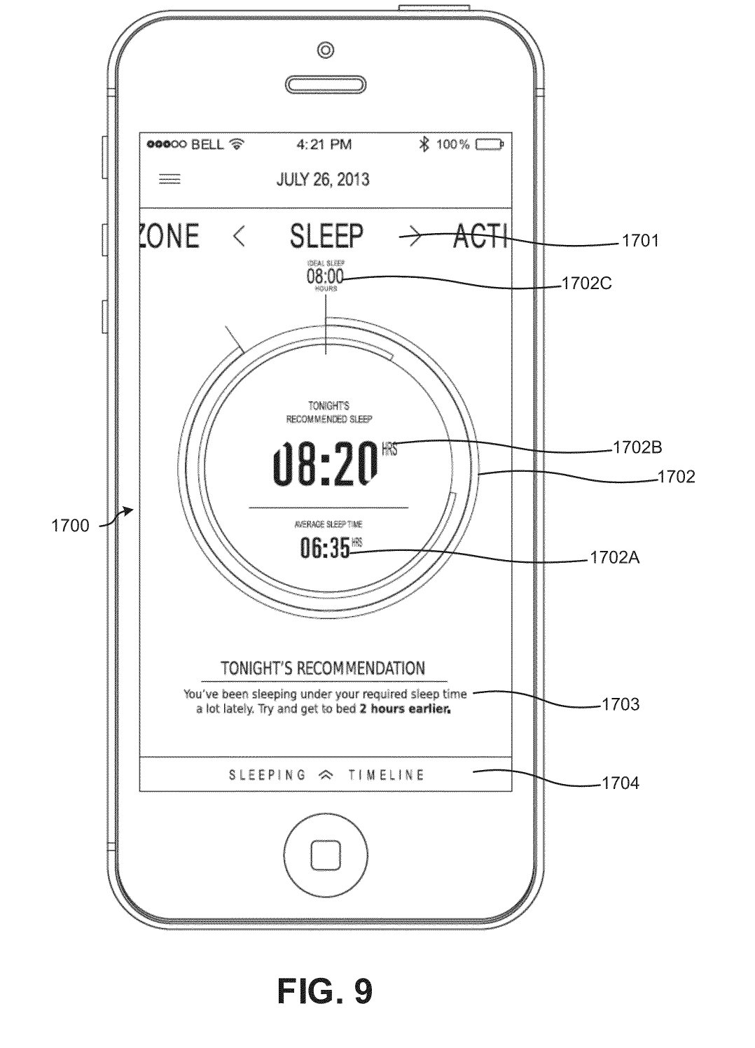

FIG. 9 illustrates a sleep display 1700 that may be associated with a sleep display module 1712. In various embodiments, sleep display 1700 may visually present to a user a record of the user's sleep history and sleep recommendations for the day. It is worth noting that in various embodiments one or more modules of the activity tracking application 1710 may automatically determine or estimate when a user is sleeping (and awake) based on an a pre-loaded or learned activity profile for sleep, in accordance with the activity profiles described above. Alternatively, the user may interact with the sleep display 1700 or other display to indicate that the current activity is sleep, enabling the system to better learn that individualized activity profile associated with sleep. The modules may also use data collected from the earphones, including fatigue level and activity score trends, to calculate a recommended amount of sleep. Systems and methods for implementing this functionality are described in greater detail in U.S. patent application Ser. No. 14/568,835, filed Dec. 12, 2014, and titled “System and Method for Creating a Dynamic Activity Profile”, and U.S. patent application Ser. No. 14/137,942, filed Dec. 20, 2013, titled “System and Method for Providing an Interpreted Recovery Score,” both of which are incorporated herein by reference in their entirety.

As illustrated, sleep display 1700 may comprise a display navigation area 1701, a center sleep display area 1702, a textual sleep recommendation 1703, and a sleeping detail or timeline 1704. Display navigation area 1701 allows a user to navigate between the various displays associated with modules 211-214 as described above. In this embodiment the sleep display 1700 includes the identification “SLEEP” at the center of the navigation area 1701.

Center sleep display area 1702 may display sleep metrics such as the user's recent average level of sleep or sleep trend 1702A, a recommended amount of sleep for the night 1702B, and an ideal average sleep amount 1702C. In various embodiments, these sleep metrics may be displayed in units of time (e.g., hours and minutes) or other suitable units. Accordingly, a user may compare a recommended sleep level for the user (e.g., metric 1702B) against the user's historical sleep level (e.g., metric 1702A). In one embodiment, the sleep metrics 1702A-1702C may be displayed as a pie chart showing the recommended and historical sleep times in different colors. In another embodiment, sleep metrics 1702A-1702C may be displayed as a curvilinear graph showing the recommended and historical sleep times as different colored, concentric lines. This particular embodiment is illustrated in example sleep display 1700, which illustrates an inner concentric line for recommended sleep metric 1702B and an outer concentric line for average sleep metric 1702A. In this example, the lines are concentric about a numerical display of the sleep metrics.

In various embodiments, a textual sleep recommendation 1703 may be displayed at the bottom or other location of display 1700 based on the user's recent sleep history. A sleeping detail or timeline 1704 may also be displayed as a collapsed bar at the bottom of sleep display 1700. In various embodiments, when a user selects sleeping detail 1704, it may display a more detailed breakdown of daily sleep metrics, including, for example, total time slept, bedtime, and wake time. In particular implementations of these embodiments, the user may edit the calculated bedtime and wake time. In additional embodiments, the selected sleeping detail 1704 may graphically display a timeline of the user's movements during the sleep hours, thereby providing an indication of how restless or restful the user's sleep is during different times, as well as the user's sleep cycles. For the example, the user's movements may be displayed as a histogram plot charting the frequency and/or intensity of movement during different sleep times.

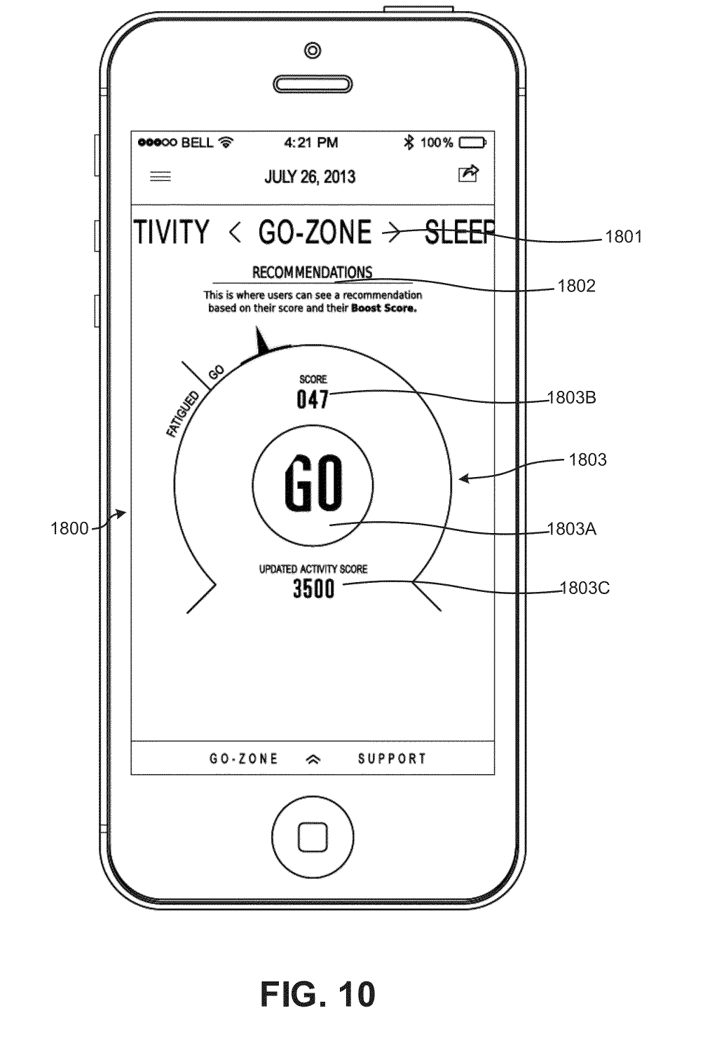

FIG. 10 illustrates an activity recommendation and fatigue level display 1800 that may be associated with an activity recommendation and fatigue level display module 213. In various embodiments, display 1800 may visually present to a user the user's current fatigue level and a recommendation of whether or not engage in activity. It is worth noting that one or more modules of activity tracking application 210 may track fatigue level based on data received from the earphones 100, and make an activity level recommendation. For example, HRV data tracked at regular intervals may be compared with other biometric or biological data to determine how fatigued the user is. Additionally, the HRV data may be compared to pre-loaded or learned fatigue level profiles, as well as a user's specified activity goals. Systems and methods for implementing this functionality are described in greater detail in U.S. patent application Ser. No. 14/140,414, filed Dec. 24, 2013, titled “System and Method for Providing an Intelligent Goal Recommendation for Activity Level”, and which is incorporated herein by reference in its entirety.

As illustrated, display 1800 may comprise a display navigation area 1801 (as described above), a textual activity recommendation 1802, and a center fatigue and activity recommendation display 1803. Textual activity recommendation 1002 may, for example, display a recommendation as to whether a user is too fatigued for activity, and thus must rest, or if the user should be active. Center display 1803 may display an indication to a user to be active (or rest) 1803A (e.g., “go”), an overall score 1803B indicating the body's overall readiness for activity, and an activity goal score 1803C indicating an activity goal for the day or other period. In various embodiments, indication 1803A may be displayed as a result of a binary decision—for example, telling the user to be active, or “go”—or on a scaled indicator—for example, a circular dial display showing that a user should be more or less active depending on where a virtual needle is pointing on the dial.

In various embodiments, display 1800 may be generated by measuring the user's HRV at the beginning of the day (e.g., within 30 minutes of waking up.) For example, the user's HRV may be automatically measured using the optical heartrate sensor 122 after the user wears the earphones in a position that generates a good signal as described in method 400. In embodiments, when the user's HRV is being measured, computing device 200 may display any one of the following: an instruction to remain relaxed while the variability in the user's heart signal (i.e., HRV) is being measured, an amount of time remaining until the HRV has been sufficiently measured, and an indication that the user's HRV is detected. After the user's HRV is measured by earphones 100 for a predetermined amount of time (e.g., two minutes), one or more processing modules of computing device 200 may determine the user's fatigue level for the day and a recommended amount of activity for the day. Activity recommendation and fatigue level display 1800 is generated based on this determination.

In further embodiments, the user's HRV may be automatically measured at predetermined intervals throughout the day using optical heartrate sensor 122. In such embodiments, activity recommendation and fatigue level display 1800 may be updated based on the updated HRV received throughout the day. In this manner, the activity recommendations presented to the user may be adjusted throughout the day.

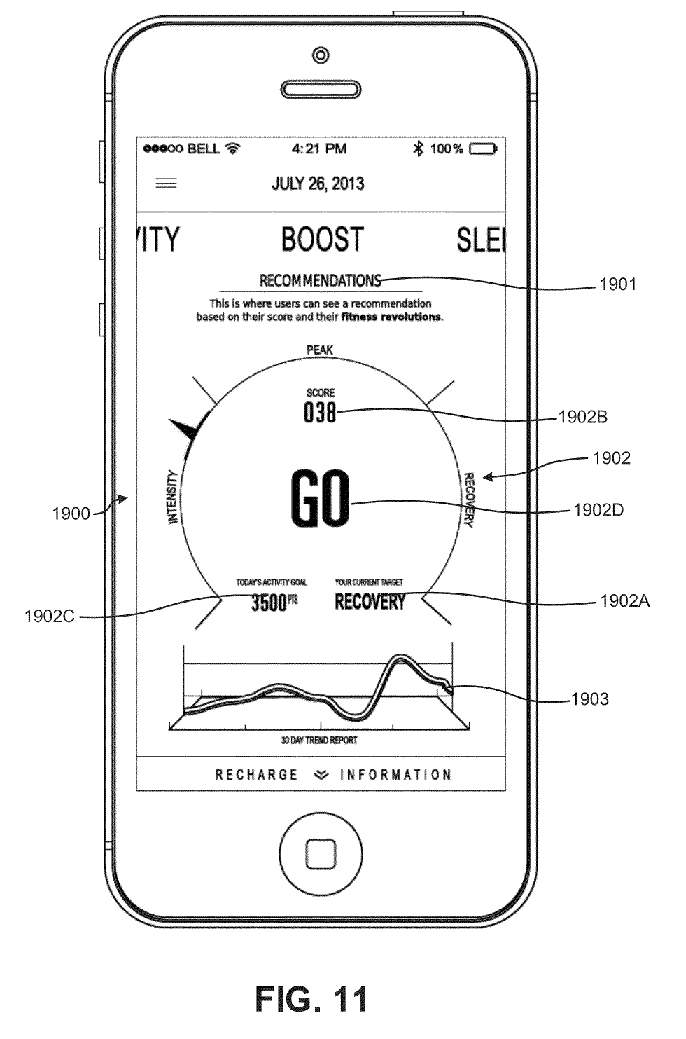

FIG. 11 illustrates a biological data and intensity recommendation display 1900 that may be associated with a biological data and intensity recommendation display module 214. In various embodiments, display 1900 may guide a user of the activity monitoring system through various fitness cycles of high-intensity activity followed by lower-intensity recovery based on the user's body fatigue and recovery level, thereby boosting the user's level of fitness and capacity on each cycle.

As illustrated, display 1900 may include a textual recommendation 1901, a center display 1902, and a historical plot 1903 indicating the user's transition between various fitness cycles. In various embodiments, textual recommendation 1901 may display a current recommended level of activity or training intensity based on current fatigue levels, current activity levels, user goals, pre-loaded profiles, activity scores, smart activity scores, historical trends, and other bio-metrics of interest. Center display 1902 may display a fitness cycle target 1902A (e.g., intensity, peak, fatigue, or recovery), an overall score 1902B indicating the body's overall readiness for activity, an activity goal score 1902C indicating an activity goal for the day or other period, and an indication to a user to be active (or rest) 1902D (e.g., “go”). The data of center display 1902 may be displayed, for example, on a virtual dial, as text, or some combination thereof. In one particular embodiment implementing a dial display, recommended transitions between various fitness cycles (e.g., intensity and recovery) may be indicated by the dial transitioning between predetermined markers.

In various embodiments, display 1900 may display a historical plot 1903 that indicates the user's historical and current transitions between various fitness cycles over a predetermined period of time (e.g., 30 days). The fitness cycles, may include, for example, a fatigue cycle, a performance cycle, and a recovery cycle. Each of these cycles may be associated with a predetermined score range (e.g., overall score 1902B). For example, in one particular implementation a fatigue cycle may be associated with an overall score range of 0 to 33, a performance cycle may be associated with an overall score range of 34 to 66, and a recovery cycle may be associated with an overall score range of 67 to 100. The transitions between the fitness cycles may be demarcated by horizontal lines intersecting the historical plot 1903 at the overall score range boundaries. For example, the illustrated historical plot 1903 includes two horizontal lines intersecting the historical plot. In this example, measurements below the lowest horizontal line indicate a first fitness cycle (e.g., fatigue cycle), measurements between the two horizontal lines indicate a second fitness cycle (e.g., performance cycle), and measurements above the highest horizontal line indicate a third fitness cycle (e.g., recovery cycle).

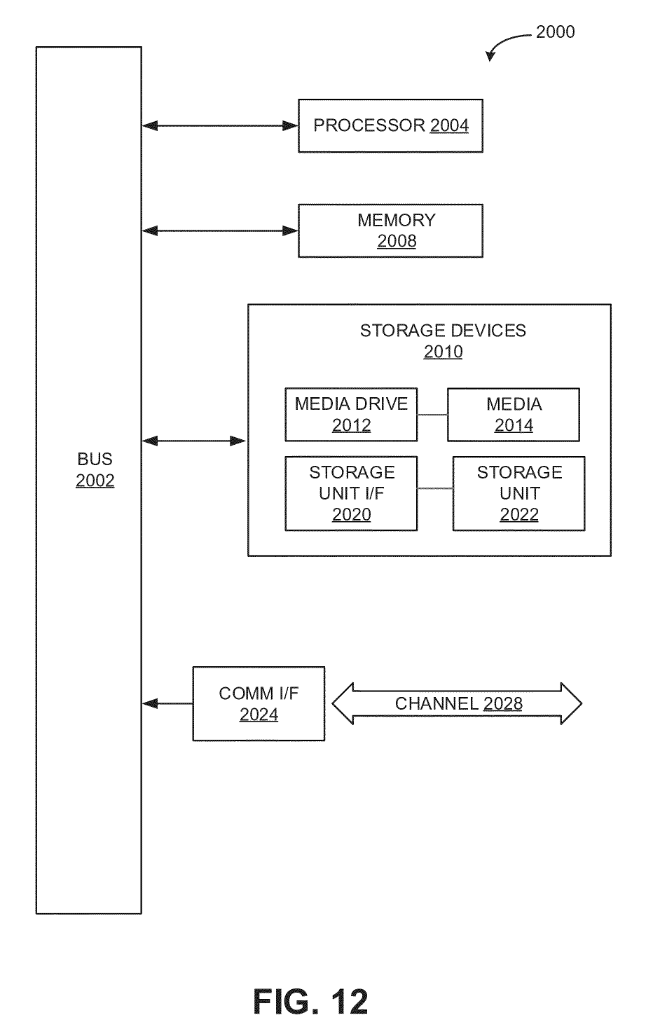

FIG. 12 illustrates an example computing module that may be used to implement various features of the systems and methods for estimating sky probes disclosed herein. As used herein, the term module might describe a given unit of functionality that can be performed in accordance with one or more embodiments of the present application. As used herein, a module might be implemented utilizing any form of hardware, software, or a combination thereof. For example, one or more processors, controllers, ASICs, PLAs, PALs, CPLDs, FPGAs, logical components, software routines or other mechanisms might be implemented to make up a module. In implementation, the various modules described herein might be implemented as discrete modules or the functions and features described can be shared in part or in total among one or more modules. In other words, as would be apparent to one of ordinary skill in the art after reading this description, the various features and functionality described herein may be implemented in any given application and can be implemented in one or more separate or shared modules in various combinations and permutations. Even though various features or elements of functionality may be individually described or claimed as separate modules, one of ordinary skill in the art will understand that these features and functionality can be shared among one or more common software and hardware elements, and such description shall not require or imply that separate hardware or software components are used to implement such features or functionality.

Where components or modules of the application are implemented in whole or in part using software, in one embodiment, these software elements can be implemented to operate with a computing or processing module capable of carrying out the functionality described with respect thereto. One such example computing module is shown in FIG. 12. Various embodiments are described in terms of this example-computing module 2000. After reading this description, it will become apparent to a person skilled in the relevant art how to implement the application using other computing modules or architectures.

Referring now to FIG. 12, computing module 2000 may represent, for example, computing or processing capabilities found within desktop, laptop, notebook, and tablet computers; hand-held computing devices (tablets, PDA's, smart phones, cell phones, palmtops, etc.); mainframes, supercomputers, workstations or servers; or any other type of special-purpose or general-purpose computing devices as may be desirable or appropriate for a given application or environment. Computing module 2000 might also represent computing capabilities embedded within or otherwise available to a given device. For example, a computing module might be found in other electronic devices such as, for example, digital cameras, navigation systems, cellular telephones, portable computing devices, modems, routers, WAPs, terminals and other electronic devices that might include some form of processing capability.

Computing module 2000 might include, for example, one or more processors, controllers, control modules, or other processing devices, such as a processor 2004. Processor 2004 might be implemented using a general-purpose or special-purpose processing engine such as, for example, a microprocessor, controller, or other control logic. In the illustrated example, processor 2004 is connected to a bus 2002, although any communication medium can be used to facilitate interaction with other components of computing module 2000 or to communicate externally.

Computing module 2000 might also include one or more memory modules, simply referred to herein as main memory 2008. For example, preferably random access memory (RAM) or other dynamic memory, might be used for storing information and instructions to be executed by processor 2004. Main memory 2008 might also be used for storing temporary variables or other intermediate information during execution of instructions to be executed by processor 2004. Computing module 2000 might likewise include a read only memory (“ROM”) or other static storage device coupled to bus 2002 for storing static information and instructions for processor 2004.

The computing module 2000 might also include one or more various forms of information storage mechanism 2010, which might include, for example, a media drive 2012 and a storage unit interface 2020. The media drive 2012 might include a drive or other mechanism to support fixed or removable storage media 2014. For example, a hard disk drive, a solid state drive, a magnetic tape drive, an optical disk drive, a CD, DVD, or Blu-ray drive (R or RW), or other removable or fixed media drive might be provided. Accordingly, storage media 2014 might include, for example, a hard disk, a solid state drive, magnetic tape, cartridge, optical disk, a CD, DVD, Blu-ray or other fixed or removable medium that is read by, written to or accessed by media drive 2012. As these examples illustrate, the storage media 2014 can include a computer usable storage medium having stored therein computer software or data.

In alternative embodiments, information storage mechanism 2010 might include other similar instrumentalities for allowing computer programs or other instructions or data to be loaded into computing module 2000. Such instrumentalities might include, for example, a fixed or removable storage unit 2022 and an interface 2020. Examples of such storage units 2022 and interfaces 2020 can include a program cartridge and cartridge interface, a removable memory (for example, a flash memory or other removable memory module) and memory slot, a PCMCIA slot and card, and other fixed or removable storage units 2022 and interfaces 2020 that allow software and data to be transferred from the storage unit 2022 to computing module 2000.

Computing module 2000 might also include a communications interface 2024. Communications interface 2024 might be used to allow software and data to be transferred between computing module 2000 and external devices. Examples of communications interface 2024 might include a modem or softmodem, a network interface (such as an Ethernet, network interface card, WiMedia, IEEE 802.XX or other interface), a communications port (such as for example, a USB port, IR port, RS232 port BLUETOOTH® interface, or other port), or other communications interface. Software and data transferred via communications interface 2024 might typically be carried on signals, which can be electronic, electromagnetic (which includes optical) or other signals capable of being exchanged by a given communications interface 2024. These signals might be provided to communications interface 2024 via a channel 2028. This channel 2028 might carry signals and might be implemented using a wired or wireless communication medium. Some examples of a channel might include a phone line, a cellular link, an RF link, an optical link, a network interface, a local or wide area network, and other wired or wireless communications channels.

In this document, the terms “computer program medium” and “computer usable medium” are used to generally refer to transitory or non-transitory media such as, for example, memory 2008, storage unit 2020, media 2014, and channel 2028. These and other various forms of computer program media or computer usable media may be involved in carrying one or more sequences of one or more instructions to a processing device for execution. Such instructions embodied on the medium, are generally referred to as “computer program code” or a “computer program product” (which may be grouped in the form of computer programs or other groupings). When executed, such instructions might enable the computing module 2000 to perform features or functions of the present application as discussed herein.

Although described above in terms of various exemplary embodiments and implementations, it should be understood that the various features, aspects and functionality described in one or more of the individual embodiments are not limited in their applicability to the particular embodiment with which they are described, but instead can be applied, alone or in various combinations, to one or more of the other embodiments of the application, whether or not such embodiments are described and whether or not such features are presented as being a part of a described embodiment. Thus, the breadth and scope of the present application should not be limited by any of the above-described exemplary embodiments.

Terms and phrases used in this document, and variations thereof, unless otherwise expressly stated, should be construed as open ended as opposed to limiting. As examples of the foregoing: the term “including” should be read as meaning “including, without limitation” or the like; the term “example” is used to provide exemplary instances of the item in discussion, not an exhaustive or limiting list thereof; the terms “a” or “an” should be read as meaning “at least one,” “one or more” or the like; and adjectives such as “conventional,” “traditional,” “normal,” “standard,” “known” and terms of similar meaning should not be construed as limiting the item described to a given time period or to an item available as of a given time, but instead should be read to encompass conventional, traditional, normal, or standard technologies that may be available or known now or at any time in the future. Likewise, where this document refers to technologies that would be apparent or known to one of ordinary skill in the art, such technologies encompass those apparent or known to the skilled artisan now or at any time in the future.

The presence of broadening words and phrases such as “one or more,” “at least,” “but not limited to” or other like phrases in some instances shall not be read to mean that the narrower case is intended or required in instances where such broadening phrases may be absent. The use of the term “module” does not imply that the components or functionality described or claimed as part of the module are all configured in a common package. Indeed, any or all of the various components of a module, whether control logic or other components, can be combined in a single package or separately maintained and can further be distributed in multiple groupings or packages or across multiple locations.

Additionally, the various embodiments set forth herein are described in terms of exemplary block diagrams, flow charts and other illustrations. As will become apparent to one of ordinary skill in the art after reading this document, the illustrated embodiments and their various alternatives can be implemented without confinement to the illustrated examples. For example, block diagrams and their accompanying description should not be construed as mandating a particular architecture or configuration.

While various embodiments of the present disclosure have been described above, it should be understood that they have been presented by way of example only, and not of limitation. Likewise, the various diagrams may depict an example architectural or other configuration for the disclosure, which is done to aid in understanding the features and functionality that can be included in the disclosure. The disclosure is not restricted to the illustrated example architectures or configurations, but the desired features can be implemented using a variety of alternative architectures and configurations. Indeed, it will be apparent to one of skill in the art how alternative functional, logical or physical partitioning and configurations can be implemented to implement the desired features of the present disclosure. Also, a multitude of different constituent module names other than those depicted herein can be applied to the various partitions. Additionally, with regard to flow diagrams, operational descriptions and method claims, the order in which the steps are presented herein shall not mandate that various embodiments be implemented to perform the recited functionality in the same order unless the context dictates otherwise.

Although the disclosure is described above in terms of various exemplary embodiments and implementations, it should be understood that the various features, aspects and functionality described in one or more of the individual embodiments are not limited in their applicability to the particular embodiment with which they are described, but instead can be applied, alone or in various combinations, to one or more of the other embodiments of the disclosure, whether or not such embodiments are described and whether or not such features are presented as being a part of a described embodiment. Thus, the breadth and scope of the present disclosure should not be limited by any of the above-described exemplary embodiments.

Terms and phrases used in this document, and variations thereof, unless otherwise expressly stated, should be construed as open ended as opposed to limiting. As examples of the foregoing: the term “including” should be read as meaning “including, without limitation” or the like; the term “example” is used to provide exemplary instances of the item in discussion, not an exhaustive or limiting list thereof; the terms “a” or “an” should be read as meaning “at least one,” “one or more” or the like; and adjectives such as “conventional,” “traditional,” “normal,” “standard,” “known” and terms of similar meaning should not be construed as limiting the item described to a given time period or to an item available as of a given time, but instead should be read to encompass conventional, traditional, normal, or standard technologies that may be available or known now or at any time in the future. Likewise, where this document refers to technologies that would be apparent or known to one of ordinary skill in the art, such technologies encompass those apparent or known to the skilled artisan now or at any time in the future.

The presence of broadening words and phrases such as “one or more,” “at least,” “but not limited to” or other like phrases in some instances shall not be read to mean that the narrower case is intended or required in instances where such broadening phrases may be absent. The use of the term “module” does not imply that the components or functionality described or claimed as part of the module are all configured in a common package. Indeed, any or all of the various components of a module, whether control logic or other components, can be combined in a single package or separately maintained and can further be distributed in multiple groupings or packages or across multiple locations.

Additionally, the various embodiments set forth herein are described in terms of exemplary block diagrams, flow charts and other illustrations. As will become apparent to one of ordinary skill in the art after reading this document, the illustrated embodiments and their various alternatives can be implemented without confinement to the illustrated examples. For example, block diagrams and their accompanying description should not be construed as mandating a particular architecture or configuration.

What is claimed is:

1. A system for tracking a user's biological age over time, comprising:
a pair of earphones comprising:
speakers;
a processor; and
a heartrate sensor electrically coupled to the processor, wherein the processor is configured to process electronic input signals from the heartrate sensor; and
a non-transitory computer-readable medium operatively coupled to at least one of one or more processors and having instructions stored thereon that, when executed by at least one of the one or more processors, cause the system to:
receive one or more user determinable biological age parameters including an actual age of the user;
determine one or more system measurable biological age parameters based on signals generated by the heartrate sensor;
calculate a biological age factor as a function of the user determinable biological age parameters and the system measurable biological age parameters;
calculate the user's biological age as a function of the biological age factor and the user's actual age; and
display on a display the user's biological age.

2. The system of claim 1, wherein the instructions, when executed by at least one of the one or more processors, further cause the system to:
receive a time interval width and desired number of time intervals for which to track the user's biological age;
calculate, for each time interval, an interval specific biological age;
store in a memory, for each time interval, the interval specific biological age;
calculate a biological age trend function based on the interval specific biological ages; and
display on the display a plot of the biological age trend function.

3. The system of claim 1, wherein the system measurable biological age parameters comprise heart rate variability, and wherein the heart rate variability is determined based on signals generated by the heartrate sensor.

4. The system of claim 3, wherein the heartrate sensor is an optical heartrate sensor protruding from a side of the earphone proximal to an interior side of a user's ear when the earphone is worn, and wherein the optical heartrate sensor is configured to measure the user's blood flow and to output an electrical signal representative of this measurement to the earphones processor.

5. The system of claim 3, wherein the pair of earphones further comprise a motion sensor, wherein the earphones processor is configured to process electronic input signals from the motion sensor, and wherein the one or more system measurable biological age parameters are determined based on signals generated by the motion sensor.

6. The system of claim 5, wherein the system measurable biological age parameters further comprise a sleep profile, an activity profile, a recovery score, an activity score, or a smart activity score.

7. The system of claim 6, wherein the instructions, when executed by at least one of the one or more processors, further cause the system to: display on the display a lifestyle trend function temporally synchronous with the biological age trend function, wherein the lifestyle trend function is based on the activity profile.

8. The system of claim 7, wherein the instructions, when executed by at least one of the one or more processors, further cause the system to:
calculate a correlation between changes in the lifestyle trend function and changes in the biological age trend function;
determine that the correlation exceeds a threshold;
based on determining that the correlation exceeds a threshold, displaying on the display an alert flag indicating that the threshold has been exceeded.

9. The system of claim 5, wherein the sleep profile comprises an average sleep quality metric and an average sleep quantity metric; and the activity profile comprises an average activity quality metric and an average activity quantity metric, and wherein the activity profile is created based on signals generated by the motion sensor.

10. The system of claim 1, wherein the user determinable biological age parameters further comprise at least two of: gender, ethnicity, eating habits, stress level, marriage profile, height, weight, body fat index, cholesterol level, blood pressure, and medical history.

11. The system of claim 10, wherein the instructions, when executed by at least one of the one or more processors, further cause the system to: determine one or more adjustments to the system measurable biological age factors and the user determinable biological age factors such that, if the adjustments are achieved, the user's biological age will match the user's actual age.

12. A method for tracking a user's biological age over time using earphones with biometric sensors, the method comprising:
receiving at a computing device one or more user determinable biological age parameters including an actual age of the user;
generating signals using a heartrate sensor of the earphones;
one or more processors determining one or more system measurable biological age parameters, wherein the one or system measurable biological age parameters comprise a heart rate variability of the user determined based on the signals generated by the heartrate sensor of the earphones;
the one or more processors calculating a biological age factor as a function of the user determinable biological age parameters and the system measurable biological age parameters;
the one or more processors calculating the user's biological age as a function of the biological age factor and the user's actual age; and
displaying on a display the user's biological age.

13. The method of claim 12, further comprising:
receiving at a user interface of the computing device a time interval width and desired number of time intervals for which to track the user's biological age;
calculating, for each time interval, an interval specific biological age;
storing in a memory, for each time interval, the interval specific biological age;
calculating a biological age trend function based on the interval specific biological ages; and
displaying on the display a plot of the biological age trend function.

14. The method of claim 12, wherein the heartrate sensor is an optical heartrate sensor protruding from a side of the earphone proximal to an interior side of a user's ear when the earphone is worn, and wherein the optical heartrate sensor is configured to measure the user's blood flow and to output an electrical signal representative of this measurement to the earphones processor.

15. The method of claim 12, further comprising: generating signals representative of the user's motion using a motion sensor of the earphones, wherein the one or more system measurable biological age parameters are determined based on the signals generated by the motion sensor.

16. The method of claim 15, wherein the system measurable biological age parameters further comprise a sleep profile, an activity profile, a recovery score, an activity score, or a smart activity score.

17. The method of claim 16, further comprising: displaying on the display a lifestyle trend function temporally synchronous with the biological age trend function, wherein the lifestyle trend function is based on the activity profile.

18. The method of claim 17, further comprising the one or more processors:
calculating a correlation between changes in the lifestyle trend function and changes in the biological age trend function;
determining that the correlation exceeds a threshold; and
based on determining that the correlation exceeds a threshold, displaying on the display an alert flag indicating that the threshold has been exceeded.

19. The method of claim 12, wherein the user determinable biological age parameters further comprise at least two of: gender, ethnicity, eating habits, stress level, marriage profile, height, weight, body fat index, cholesterol level, blood pressure, and medical history.

20. The method of claim 19, further comprising: the one or more processors determining one or more adjustments to the system measurable biological age factors and the user determinable biological age factors such that, if the adjustments are achieved, the user's biological age will match the user's actual age.
